(19) United States
(12) Patent Application Publication
(10) Pub. No.: US 2026/0021269 A1
(43) Pub. Date: Jan. 22, 2026

(54) Dental Anesthesia Delivery Device and Method of Use

(71) Applicant: Robert Harrell, Charlotte, NC (US)

(72) Inventor: Robert Harrell, Charlotte, NC (US)

(21) Appl. No.: 19/271,030

(22) Filed: Jul. 16, 2025

Related U.S. Application Data
(60) Provisional application No. 63/672,473, filed on Jul. 17, 2024.

(51) Int. Cl. A61M 19/00; A61C 1/08; A61M 5/32

(52) U.S. Cl. CPC A61M 19/00; A61C 1/08; A61M 5/32

(57) ABSTRACT

A device and method for perforating human gingeva and cortical plate tissue to allow for the administration of dental anesthesia directly to the nerve(s) of a human mandibular molar or molars.

### CROSS REFERENCE TO RELATED APPLICATIONS

This application claims priority to U.S. provisional application 63/672,473, dated Jul. 17, 2024.

### STATEMENT REGARDING FEDERALLY SPONSORED RESEARCH OR DEVELOPMENT

No federal government funds were used in researching or developing this invention.

### NAMES OF PARTIES TO A JOINT RESEARCH AGREEMENT

Not applicable.

### SEQUENCE LISTING INCLUDED AND INCORPORATED BY REFERENCE HEREIN

Not applicable.

### BACKGROUND

### Field of the Invention

The invention is a device and method of use for administering dental anesthesia directly to the nerve(s) of a specific tooth or teeth.

### Background of the Invention

Intraosseous topical anesthesia delivery systems for use in dental procedures are long known. See, e.g. the Stabident Intraosseous System currently offered by Henry Schein, Inc.

Such intraosseous delivery systems generally involve first anaesthetizing the soft tissue or gingiva surrounding the tooth, then perforating the cortical plate of the bone before finally injecting anesthetic directly into the cancellous bone space around the tooth.

Upon injection of anesthesia into cancellous bone, pulpal numbing occurs and work can begin on the affected tooth immediately. Known systems have successfully decreased the pain experienced by patients during dental procedures, as well as the aftereffects of numbness and tissue swelling.

However, known systems rely on on needles for perforation, which can be overly fragile and difficult to employ in the penetration of bone. Such needle-based systems can result in procedures that require multiple attempts at perforation, with scraping or dragging of the needle against bone. Such difficulties are not only uncomfortable for the patient in the moment, but can exacerbate swelling and post-procedure soreness. In a worst case scenario, the narrow gauge needle can actually deform or snap during a perforation attempt. What is needed is a more robust perforation device that maintains a minimal level of invasiveness in introducing intraosseous anesthesia without sacrificing patient comfort.

### BRIEF DESCRIPTION OF THE INVENTION

In a preferred embodiment, a system for numbing one or more human molars prior to performing a dental procedure, comprising the components:
(1) a dental hypodermic needle; and
(2) a dental perforating device for perforating a dental patient's gingiva and cortical plate, such dental perforation device comprising a single metallic rod with a first side and a second side, wherein the first side is a small gauge drill stem comprising drill threads and a drill tip and the second side comprises a drill shank with a drill connector, such first and second sides separated by a stopper disc circumnavigating the rod;
such system components executing the following steps:
(a) Numbing a dental patient's gingiva by injecting anesthetic with a dental hypodermic needle and anesthetic;
(b) Perforating and creating a perforation channel through the gingiva and into the dental patient's cortical plate with the dental perforating device; and
(c) Injecting anesthetic into the cortical plate through the perforation channel at the roots of a mandibular molar or molars adjacent to the perforation channel to achieve immediate pulpal anesthesia of the mandibular molar or molars.

In another preferred embodiment, the system as described herein, wherein the dental perforating device is powered by a mechanical drill assembly.

In another preferred embodiment, the system as described herein, wherein the stopper disc is integrated with the metallic rod.

In another preferred embodiment, the system as described herein, wherein the stopper disc is adhered to the metallic rod.

In an alternate preferred embodiment, a method of use of the dental perforating device of claim 2, comprising the steps of: perforating and creating an perforation channel through a patient's gingiva and into the patient's cortical plate using the dental perforating device; and injecting anesthetic into the cortical plate through the perforation channel at the roots of a mandibular molar or molars adjacent to the perforation channel to achieve immediate pulpal anesthesia of the mandibular molar or molars.

In another preferred embodiment, the method of use as described herein, wherein the dental perforation device enters the cortical plate approximately 5-6 mm below a crest of bone within a divergence of proximal roots of the mandibular molar or molars.

### BRIEF DESCRIPTION OF THE DRAWINGS

FIG. 1 shows a side view the inventive dental perforating device.

FIG. 2 shows a perspective view the inventive dental perforating device.

FIG. 3 shows the dental perforating device of FIGS. 1-2 perforating a dental patient's jawbone through the gumline.

FIG. 4 shows the insertion of a hypodermic needle into the perforation made by the dental perforating device in FIG. 3.

### DETAILED DESCRIPTION OF THE INVENTION

The invention comprises a dental perforating device designed to be used as a bone drill or bone perforator specifically in the human posterior mandible. The device comprises components as described in the detailed description of the figures, which also include certain preferable specifications. However, specifications such as length, gauge and configuration are subject to adjustment based upon the type of patient and other factors for a specific usage.

The cortical plate is a very dense bone, making it hard to penetrate. Known systems are surprisingly rudimentary and use a needle embodied as a beveled metal rod which is not designed for cutting or puncturing bone and is thus prone to deformation once it penetrates the gingiva and reaches the cortical plate. The needles are known to bend and break at the point of entry, and also often fail to achieve sufficient penetration to fully numb the relevant molars.

Known In a preferred method of use, the operator uses the perforating device to perforate first the oral soft tissue attached gingiva (gums) and thence the cortical plate adjacent to the teeth to be numbed. Once the perforation is achieved the operator may inject dental anesthesia directly into the bone perforation orifice with a standard needle, such as lidocaine or a similar topical agent, to achieve immediate pulpal anesthesia of the mandibular molars. This device allows for quick and atraumatic perforation to prevent tissue damage from drill friction.

The point of perforation through the gingeva is approximately 5-6 mm below the crest of bone within the divergence of the proximal roots of mandibular molars.

Preferably, the device is machined from a stainless steel rod specs SS420 J2. However, other metals, alloys or materials accepted for use in dental practice may be substituted.

### DETAILED DESCRIPTION OF THE FIGURES

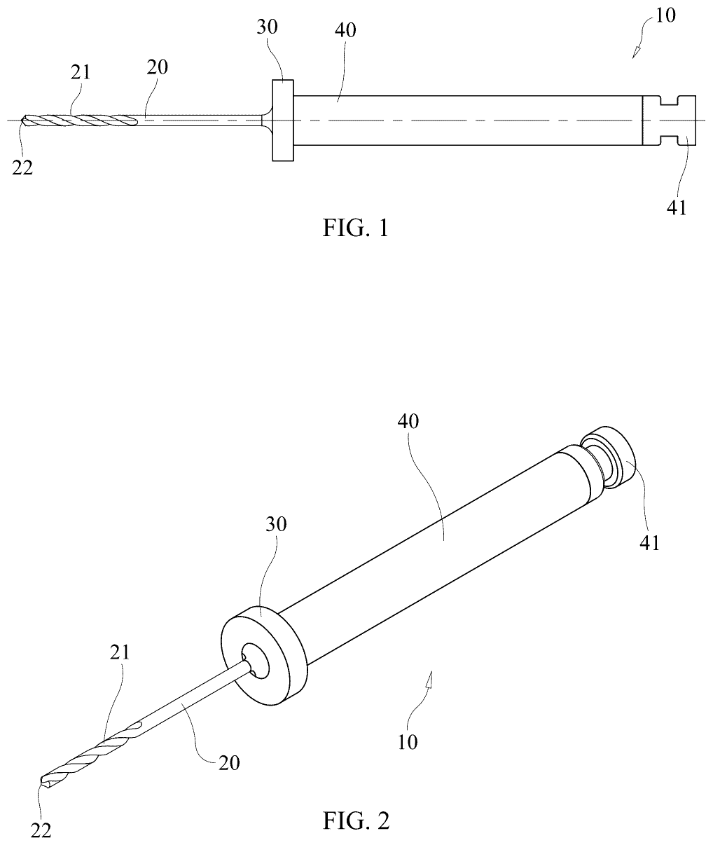

FIG. 1 shows a side view of the inventive dental perforating device 10, evidencing a first side as a drill stem 20 with threads 21 to be used in perforating both the gingiva and cortical plate (bone) adjacent to the tooth or teeth to be operated upon. The tip of the drill stem comprises the drill tip 22, which will be the tip perforating patient tissues. A second side of the device comprises a drill shank 40, ending in a drill connector 41, which will be attached to a power drill. The drill connector may be configured in any manner to yield a reliable connection with the drill, including but not limited to a tang shank, three- or five-flat shank, a reduced shank, a slotted shank or any similarly appropriate design. Last, the first and second sides of the device are optionally separated by a stopper ring 30, embodied as a disc either integrated with the device or adhered thereto. The stopper ring will preferably be comprised of the same material as the rest of the device but, if adhered, may be comprised of a second type of material.

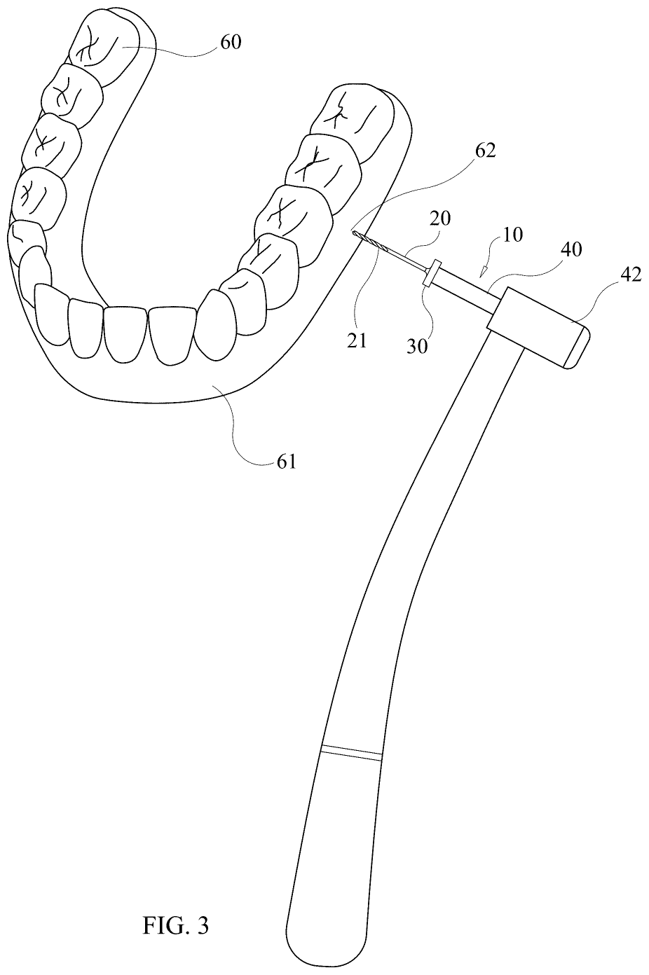

FIG. 2 shows a perspective view of the dental perforating device 10 of FIG. 1, showing the drill connector 41 embodied as a slotted tang.

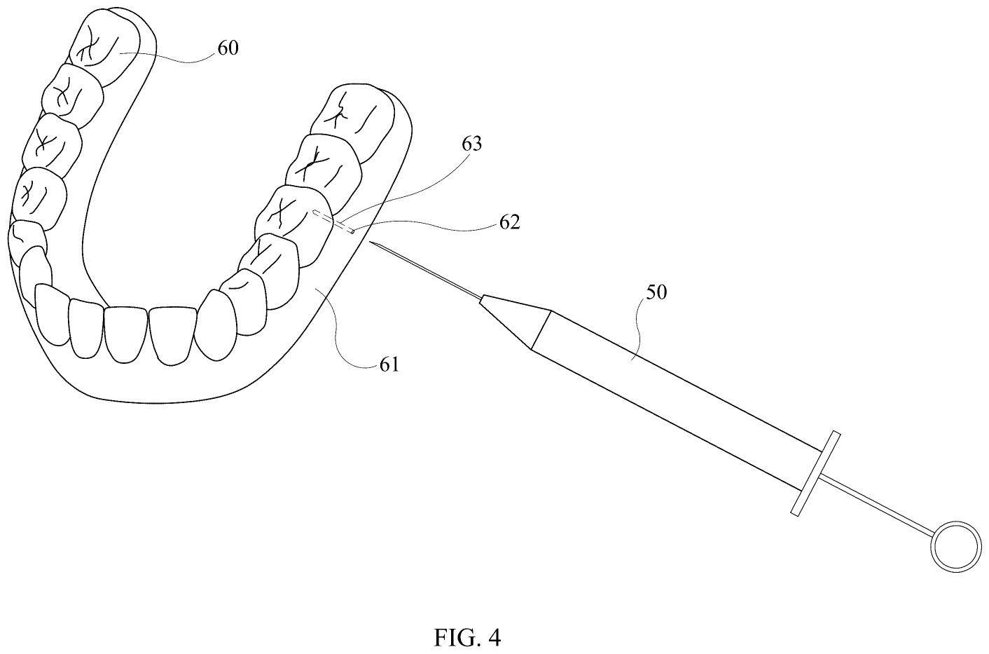

FIG. 3 shows the dental perforating device 10 employed facing a dental patient's teeth 60 and gingeva 62. The device is attached to a mechanical drill assembly 42 with drill connector 41 (inside the assembly, not pictured) and is thus operational. Drill shank 40 emanates from the drill assembly and attaches to stopper ring 30, thence to drill stem 20, drill threads 21 and finally to drill tip 22. The drill tip is not pictured as it has entered the patient's gingeva 61 at perforation point 62 and will proceed through the gum and into the patient's cortical plate (not pictured).

In a preferred embodiment, the point of perforation through the gingeva is approximately 5-6 mm below the crest of bone within the divergence of the proximal roots of mandibular molars.

FIG. 4 shows a dental patient's teeth 60 and gum 61 after the perforation step shown in FIG. 3. The perforation at perforation point 62 has resulted in a perforation channel 63 leading through the gingeva and into the patient's cortical plate (under the gingeva, not pictured). In FIG. 4, a hypodermic needle 50 is being inserted through the perforation point and into the perforation channel to allow an injection of numbing medication directly into the patient's cortical plate, thus numbing one or more teeth prior to a dental procedure.

### PART NUMBERS

10 Dental perforating device
20 Drill stem
21 Drill threads
22 Drill tip
30 Stopper ring
40 Drill shank
41 Drill connector
42 Mechanical drill assembly
50 Hypodermic needle
60 Patient teeth
61 Patient gingeva
62 Perforation point
63 Perforation channel

Unless indicated otherwise, identical reference numbers in the figures identify identical components with the same function. The terms drive unit and drive are used interchangeably herein.

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention. Accordingly, the scope of the invention is determined by the scope of the following claims and their equitable equivalents.

We claim:

1. A system for numbing one or more human molars prior to performing a dental procedure, comprising the components:
(1) a dental hypodermic needle; and
(2) a dental perforating device for perforating a dental patient's gingiva and cortical plate, such dental perforation device comprising a single metallic rod with a first side and a second side, wherein the first side is a small gauge drill stem comprising drill threads and a drill tip and the second side comprises a drill shank with a drill connector, such first and second sides separated by a stopper disc circumnavigating the rod;
such system components executing the following steps:
a. Numbing a dental patient's gingiva by injecting anesthetic with a dental hypodermic needle and anesthetic;
b. Perforating and creating a perforation channel through the gingiva and into the dental patient's cortical plate with the dental perforating device; and
c. Injecting anesthetic into the cortical plate through the perforation channel at the roots of a mandibular molar or molars adjacent to the perforation channel to achieve immediate pulpal anesthesia of the mandibular molar or molars.

2. The system of claim 1, wherein the dental perforating device is powered by a mechanical drill assembly.

3. The system of claim 1, wherein the stopper disc is integrated with the metallic rod.

4. The system of claim 1, wherein the stopper disc is adhered to the metallic rod.

5. A method of use of the dental perforating device of claim 2, comprising the steps of:
a. Perforating and creating an perforation channel through a patient's gingiva and into the patient's cortical plate using the dental perforating device; and
b. Injecting anesthetic into the cortical plate through the perforation channel at the roots of a mandibular molar or molars adjacent to the perforation channel to achieve immediate pulpal anesthesia of the mandibular molar or molars.

6. The method of claim 5, wherein the dental perforation device enters the cortical plate approximately 5-6 mm below a crest of bone within a divergence of proximal roots of the mandibular molar or molars.
